(19) United States
(12) Patent Application Publication
(10) Pub. No.: US 2004/0013273 A1
(43) Pub. Date: Jan. 22, 2004

(54) Calibration for an active noise control system

(75) Inventors: Manish Vaishya, Auburn Hills, MI (US); Paul D. Daly, Troy, MI (US)

Correspondence Address: SIEMENS CORPORATION, INTELLECTUAL PROPERTY LAW DEPARTMENT, 170 WOOD AVENUE SOUTH, ISELIN, NJ, 08830, US

(73) Assignee: Siemens VDO Automotive, Inc.

(21) Appl. No.: 10/606,981

(22) Filed: Jun. 26, 2003

Related U.S. Application Data
(60) Provisional application No. 60/397,709, filed on Jul. 22, 2002.

(51) Int. Cl. H04R 29/00; A61F 11/06; G10K 11/16; H03B 29/00

(52) U.S. Cl. 381/58; 381/71.2

(57) ABSTRACT

An active noise control system (20) includes a controller (26) that drives a speaker (28) to cancel out or reduce noise. A microphone (30) provides information to the controller (26) regarding the system response to the noise source sound and the cancellation signal. The controller (26) uses a selected noise source sound as a calibration reference. By subsequently comparing the system response to the same sound, the controller (26) is able to calibrate the system and make any adjustments necessary to account for microphone drift, for example.

### CROSS REFERENCE TO RELATED APPLICATIONS

This application claims priority to Provisional Application No. 60/397,709, which was filed on Jul. 22, 2002.

### FIELD OF THE INVENTION

This invention generally relates to active noise control systems. More particularly, this invention relates to calibrating an active noise control system.

2. Description of the Related Art

Noise control systems are currently used in a variety of circumstances including on automotive vehicles for reducing noise propagation into the passenger compartment. For example, the air induction system of a vehicle can propagate engine noise in a manner that makes it noticeable within a passenger compartment. Various efforts have been made to reduce the amount of engine noise traveling through the air induction system. Some arrangements include passive devices such as expansion chambers and Helmholtz resonators. Active noise control systems have also been used for this purpose.

Typical activate noise control systems include a speaker that generates a sound to attenuate the noise that is to be reduced or cancelled. The sound from the speaker typically is out of phase with the sound from the noise source. The two sounds combine such that the result is a reduced or enhanced sound, which results in less noise transmission into the passenger compartment, for example. The speaker sound can be referred to as a noise cancellation signal.

In such active systems, calibration is achieved by applying a gain to the microphone signal that indicates the resulting sound (i.e., an error signal) of the combination of the noise and the cancellation signal. There are disadvantages to this approach because it is not capable of addressing engine noise irregularities. Additionally, if there is a weak signal-to-noise ratio, the system can become unstable. This is because the sound at certain orders of low engine noise typically do not provide a sufficiently consistent or detectable signal for generating an error signal. Additionally, typical microphones that are acceptable in terms of cost and operation for such systems are not capable of a detection range that encompasses all engine sounds.

It is desirable to provide better controls for active noise control systems. With the introduction of new controls, there is a need for different calibration techniques. This invention provides a new calibration technique for active noise control systems.

### SUMMARY OF THE INVENTION

In general terms, this invention is a method of calibrating an active noise control system that includes selecting at least one noise source sound as a calibration reference. For systems where the noise source is an engine, at least one selected engine sound serves as the calibration reference.

In one example method designed according to this invention, a harmonic representation of the system response to the selected sound is determined and used as the calibration reference. During subsequent system operation, a harmonic representation of the actual system response to the same sound is determined and a comparison between that and the calibration reference provides information regarding any needed calibration of the system.

An example system designed according to this invention includes a microphone and a speaker. A controller drives the speaker to selectively generate a noise cancellation signal. The controller interprets the signal from the microphone indicating a resulting system response to a noise source sound, the noise cancellation signal or a combination of them. The controller uses at least one noise source sound as a calibration reference.

The various features and advantages of this invention will become apparent to those skilled in the art from the following detailed description of the currently preferred embodiment. The drawings that accompany the detailed description can be briefly described as follows.

### BRIEF DESCRIPTION OF THE DRAWINGS

FIG. 1 schematically illustrates a vehicle including a noise control system designed according to this invention.

FIG. 2 schematically illustrates a control strategy useful with a noise control system designed according to this invention.

### DETAILED DESCRIPTION OF THE PREFERRED EMBODIMENT

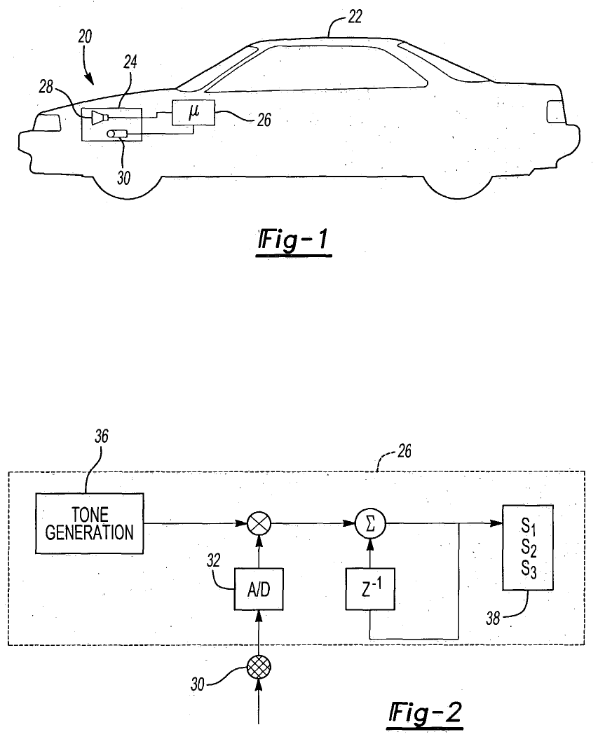

FIG. 1 schematically illustrates a noise control system 20. In this example, the noise control system 20 is provided on a vehicle 22 for controlling engine noise propagation through an air intake 24 associated with the vehicle engine (not illustrated). The air intake 24 comprises conventional components.

The noise control system 20 includes a controller 26 that drives a speaker 28 to generate a noise cancellation signal to reduce or cancel out noise from the air intake 24 to control the amount of noise propagation into the passenger compartment of the vehicle 22. A microphone 30 provides information to the controller 26 regarding the sounds within the air intake 24 so that the controller 26 can, for example, calibrate the system to insure desired noise cancellation performance.

In one example, the noise control system 20 utilizes a sound pressure level specification to achieve a desired noise control. One approach that uses a sound pressure level specification is disclosed in U.S. patent application Ser. No. 10/339,539, which was filed on Jan. 9, 2003. The teachings of that specification are incorporated into this description by reference. In one example, an absolute sound pressure level is specified for weak engine orders at each engine RPM. The require sound pressure level constitutes a desired signal fed to the system microphone. The generated tones are amplified by the required sound pressure level and the analog-to-digital gain. These tones are summed and provided to the error microphone.

Using an absolute sound pressure level specification has advantages compared to prior noise control systems that merely apply a gain to a microphone signal to obtain a desired voltage level for a cancellation signal. The sound pressure level specification technique allows the noise control system to achieve a sound regardless of what the noise source (i.e., the vehicle engine) produces. Additionally, the sound pressure level specification technique avoids any difficulties previously associated with weak signal-to-noise ratio conditions.

According to this invention, at least one sound from the noise source is chosen as a calibration reference. In the illustrated example, at least one engine sound is chosen for the calibration reference. In one example, a plurality of engine sounds, each at a dominant order, are chosen as calibration references. The dominant orders typically are a multiple of the number of cylinders associated with a vehicle engine. In one example, an engine sound at an order corresponding to one-half the number of engine cylinders is chosen as the calibration reference.

The controller 26 uses the calibration reference sound to calibrate the system to accommodate for any microphone drift or other irregularities that occur in the noise control system over time.

The selected calibration sound evokes a system response, which is detected using the microphone 30. The controller stores a harmonic representation of that response as a set of calibration values.

In one example, the controller 26 is programmed to calibrate the system often. Those skilled in the art who have the benefit of this description will be able to select an appropriate timing for calibration. According to one example, the controller 26 receives a signal from the microphone 30 indicating the response of the noise control system 20 (i.e., the sound within the air intake 24) when the noise source produces the selected calibration sound. The controller 26 converts the microphone signal into a harmonic representation of the measured sound. The harmonic representation is then compared to the stored values representing the harmonic representation of the expected system response to the calibration reference sound. Because the controller 26 knows what the system response should be, comparing the actual system response to the expected system response provides the controller 26 with sufficient information to calibrate the system and to make any adjustments that may be needed.

FIG. 2 schematically demonstrates one control strategy designed according to this invention. Assuming that the noise control feature of the active noise control system 20 is not active, calibration can be carried out by measuring the engine sound spectra and mapping those sounds for different throttle values and engine speeds. In one example, the resulting sounds (i.e., the error signals) are decomposed using Fourier transforms to obtain a harmonic representation of the sounds.

In another example, quadrature tones are artificially generated that correspond to the calibration orders. Because such quadrature tones are orthogonal, the dot product with the error signal yields the individual Fourier coefficients when integrated over time. As shown schematically in FIG. 2, the microphone 30 provides a signal (i.e., the system response) to the controller 26. The microphone signal is processed by an analog-to-digital converter portion 32. The digital representation is then combined with the artificially generated quadrature tones, which are provided by a tone generation module 36 within the controller 26. The dot product of the microphone signal and the quadrature tones are integrated over time to yield Fourier coefficients at 38. These values then are compared to the reference values obtained from the calibration references (i.e., the selected engine sounds and corresponding system response information already stored in the controller 26).

In one example, the microphone gain H (ω) can be obtained from the ratio of S₁, S₂, etc., (FIG. 2) and the predetermined known sound pressures N₁, N₂, etc., under those conditions. Accordingly, the microphone gain can be represented by the equation: Hᵢ (ω)=|Sᵢ/Nᵢ|. This equation provides the microphone gain in terms of volts per pascal because the microphone readings Sᵢ are in terms of volts and the calibration base reference numbers Nᵢ are provided in terms of pascals.

As the engine speed (or other noise source performance variable) changes, ω spans the whole frequency range and a complete frequency-domain transfer function F(ω) is obtainable. Such mapping can be done over a number of different throttle and engine speed values.

The following table illustrates an example calibration format providing sound pressure data (i.e., RMS values) for the n^{th} order.

| RPM | | | |
|---|---|---|---|
| Throttle | 3000 | 4000 | 5000 |
| 60% | Nₙ₁₁ | Nₙ₁₂ | Nₙ₁₃ |
| 80% | Nₙ₂₁ | Nₙ₂₂ | Nₙ₂₃ |
| 100% | Nₙ₃₁ | Nₙ₃₂ | Nₙ₃₃ |

In a situation where the active noise control is on and perfect noise cancellation is assumed, the error signal (i.e., the system response obtained from the microphone 30) tends toward zero. Under such circumstances, according to one example, the engine sound is estimated to be the exact inverse of the noise cancellation signal produced by the system 20. For normalized, filtered-X least mean squares (FXLMS) algorithms, this implies that the eigen values are the same as the amplitude of the input signal (i.e., the induction sound). The microphone calibration for such a condition can be represented by the following equation:

Hₙ(ω)=\sqrt{Aₙ²+A_{n+N}²}+/Nₙ

Where Nₙ is the engine sound for orders n and A is the tap value from the control software within the controller 26 (i.e., the main FXLMS algorithm). When this approach is used, the slowly changing values (A-tap values) need to be monitored, which is readily accomplished. This is advantageous compared to trying to monitor the high frequency signal of the induction sound. The only expected resulting error in this approach is the path modeling error in the system, which is usually small.

There are circumstances where perfect cancellation cannot be assumed and there is some residual error in the system. This system can be described by the following equations:

X_{d}−X_{c}=ε

L=20×log₁₀((X_{d}−X_{c})/X_{d})

Here, X_{d} is the desired signal (the actual induction sound), X_{c} is the controller output and L is the cancellation achieved. If E is the calibration error resulting from ignoring the error signal, then the relationship between L and E is given by the following equation:

E=20×log₁₀(1−10^{L/20})

Those skilled in the art will recognize that under many circumstances the resulting error from ignoring the residual error in the system during calibration will be acceptable. For example, if the cancellation is 12 dB, then the resulting error is about 2.5 dB, which is acceptable under certain circumstances. It should be noted, however, that if the system is not fully converged and noise cancellation is poor, the errors may tend to increase.

Another technique designed according to this invention that is useful for situations where the active noise control is on includes accounting for residual error in the system. In one example, the residual error is extracted from the induction sound based upon the total error signal. In this example, the control signal is subtracted from the error signal (i.e., the resulting sound from the microphone) and the difference is decomposed into individual orders. In one example, performing the subtraction first provides a better result. The microphone gain in this example can be described by the following equation:

Hₙ(ω)=(TₙK_{0,n}AₙP+TₙK_{0,n}A_{n+N}P−ε)/Nₙ

Basically, the microphone signal provides a measurement of the residual error. Because the controller 26 knows what sound was produced by the speaker 28, the controller 26 can subtract the produced noise cancellation signal (i.e., the speaker sound) from the measured microphone sound and determine the engine sound at that time.

Where Tₙ is the tone for the nth order, K₀ is the normalizing coefficient for the plant model, and P is the transfer function of the actual path. Computing the value for Hₙ in the actual system in this example includes replacing the quantity P by the plant model. The product term TₙK_{0,n}C is available from the FXLMS control algorithm, which is the normalized reference tone that goes into the update equation. Accordingly, with this technique, the only additional computation required is a simple product of this term with Aₙ. Subsequently this value is subtracted from the error signal. The individual orders are then computed in the same manner described above for when there is no active noise cancellation.

This invention provides a reliable way of calibrating an active noise control system. This invention is especially useful for active noise control systems that rely upon absolute sound pressure level specification for generating a noise cancellation signal.

This invention includes the advantage of eliminating the need for an expensive calibrated microphone. Relying upon a known noise source sound as a calibration reference avoids the difficulties associated with other calibration techniques and eliminates the need for expensive microphone components, which are not practical for many situations.

The inventive calibration technique is less sensitive to plant modeling errors than other methods that employ direct gain to engine sounds. Further, this invention has enhanced capability to control weak orders. This invention is also less sensitive to engine sound variability at the calibration orders and is completely insensitive to other orders that are not selected as calibration references.

The preceding description is exemplary rather than limiting in nature. Variations and modifications to the disclosed examples may become apparent to those skilled in the art that do not necessarily depart from the essence of this invention. The scope of legal protection given to this invention can only be determined by studying the following claims.

We claim:

1. A method of calibrating an active noise control system, comprising:
selecting at least one noise source sound as a calibration reference.

2. The method of claim 1, including selecting a plurality of dominant order noise source sounds.

3. The method of claim 1, including determining the system response to the sound, determining a harmonic representation of the determined response and using the determined harmonic representation as the calibration reference.

4. The method of claim 3, including subsequently determining an actual harmonic representation of the system response to the same sound and determining whether the actual harmonic representation corresponds to the calibration reference.

5. The method of claim 4, wherein the system response comprises a microphone signal indicative of a sound detected by the microphone.

6. The method of claim 3, wherein the noise source is a vehicle engine and including determining the harmonic representation at a plurality of engine speeds and a plurality of throttle conditions.

7. The method of claim 1, wherein the noise source is a vehicle engine having a number of cylinders and the selected sound is from a dominant order which is a factor applied to the number of cylinders.

8. The method of claim 7, wherein the factor is ½.

9. The method of claim 1, including estimating the noise source sound as an inverse of a produced cancellation signal.

10. The method of claim 1, including estimating the noise source sound as the difference between a system response to the noise source sound and a produced cancellation signal.

11. A noise control system, comprising:
a microphone that detects a sound;
a speaker; and
a controller that drives the speaker to selectively generate a noise cancellation signal and interprets a signal from the microphone indicating a resulting system response to a combination of a noise source sound and the noise cancellation signal, the controller using at least one noise source sound as a calibration reference.

12. The system of claim 11, wherein the controller uses a plurality of dominant order noise source sounds.

13. The system of claim 11, wherein the controller determines the system response and a harmonic representation of the determined response, the controller using the determined harmonic representation as the calibration reference.

14. The system of claim 13, wherein the controller determines an actual harmonic representation of the system response at a selected time and determines whether the actual harmonic representation corresponds to the calibration reference.

15. The system of claim 13, wherein the noise source is a vehicle engine and the controller determines the harmonic representation at a plurality of engine speeds and a plurality of throttle conditions.

16. The system of claim 11, wherein the noise source is a vehicle engine having a number of cylinders and the calibration reference sound is from a dominant order which is a factor applied to the number of cylinders.

17. The system of claim 16, wherein the factor is ½.

18. The system of claim 11, wherein the controller estimates the noise source sound as an inverse of a produced cancellation signal.

19. The system of claim 11, wherein the controller estimates the noise source sound as the difference between the system response to the noise source sound and a produced cancellation signal.

20. The system of claim 19, wherein the system response comprises a signal from the microphone.
